SEQUENCE LISTING (3 sequences)

SEQ ID NO: 1 - DNA, 5740 bp, Artificial Sequence
  pNO5c with Lactobacillus kefir -ADH
aattcttaagaaggagatatacatatgactgatcgtttaaaaggcaaagtagcaattgtaactggcggtaccttgggaattggcttggcaatcgctgataagtttgttgaagaaggcgcaaaggttgttattaccggccgtcacgctgatgtaggtgaaaaagctgccaaatcaatcggcggcacagacgttatccgttttgtccaacacgatgcttctgatgaagccggctggactaagttgtttgatacgactgaagaagcatttggcccagttaccacggttgtcaacaatgccggaattgcggtcagcaagagtgttgaagataccacaactgaagaatggcgcaagctgctctcagttaacttggatggtgtcttcttcggtacccgtcttggaatccaacgtatgaagaataaaggactcggagcatcaatcatcaatatgtcatctatcgaaggttttgttggtgatccaactctgggtgcatacaacgcttcaaaaggtgctgtcagaattatgtctaaatcagctgccttggattgcgctttgaaggactacgatgttcgggttaacactgttcatccaggttatatcaagacaccattggttgacgatcttgaaggggcagaagaaatgatgtcacagcggaccaagacaccaatgggtcatatcggtgaacctaacgatatcgcttggatctgtgtttacctggcatctgacgaatctaaatttgccactggtgcagaattcgttgtcgatggtggatacactgctcaataaggatccgaattcgagctcaggaggtatcatatgcagccaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggattagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaac

SEQ ID NO: 2 - DNA, 5341 bp, Artifical Sequence
  pNO8c with Thermoplasma acidophilum - GDH
tatgaccgagcagaaagcgattgtgaccgatgcgccgaaaggtggtgtgaaatacaccaccattgatatgccggaaccggaacattatgatgcgaaactgagcccggtgtatatcggtatttgcggcaccgatcgtggtgaagtggcgggtgcgctgagctttacctataacccggaaggcgaaaactttctggtgctgggccatgaagcgctgctgcgtgtggatgatgcgcgtgataacggctatatcaaaaagggcgatctggtggtgccgctggtgcgtcgtccgggtaaatgcattaactgccgcattggccgtcaggataactgtagcattggcgatccggataaacatgaagcgggcattaccggcctgcatggctttatgcgcgatgtgatctatgatgatattgaatatctggtgaaagtggaagatccggaactgggtcgtattgcggtgctgaccgaaccgctgaaaaacgtgatgaaagcgtttgaagtgtttgatgtggtgagcaaacgcagcattttctttggcgatgatagcaccctgattggcaaacgcatggtgattatcggcagcggtagcgaagcgtttctgtatagctttgcgggcgtggatcgtggttttgatgtgaccatggtgaaccgccatgatgaaaccgaaaacaaactgaaaatcatggatgaatttggcgtgaaattcgcgaactatctgaaagatatgccggagaaaatcgatctgctggttgataccagcggtgatccgaccaccaccttcaaattcctgcgcaaagtgaacaacaacggcgtggtgattctgtttggcaccaacggtaaagcgccgggttatccggtggatggcgaagatattgattacattgtggaacgcaacattaccattgcgggtagcgtggatgcggcgaaaatccattatgtgcaggcgctgcaaagcctgagcaactggaatcgtcgtcatccggatgcgatgaaaagcattatcacctatgaagcgaaaccgagcgaaaccaacattttctttcagaaaccgcatggcgaaatcaaaaccgtgatcaaatggcagtaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtataccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaacaattcttaagaaggagatataca

SEQ ID NO: 3 - DNA, 6269 bp, Artificial Sequence
  pNO14c with R. erythropolis - sADH and B. subtilis - GDH
tatgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggttaatagtagaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtataggcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaacaattcttaagaaggagatatacatatgaaagcgatccagtacacccgtattggtgcggaaccggaactgaccgaaatcccgaaaccggaaccgggtccgggtgaagttctgctggaagttaccgcggcgggtgtttgtcatagcgatgatttcatcatgagcctgccggaagaacagtatacctatggcctgccgctgaccctgggtcatgaaggtgcgggtaaagttgcggcggttggcgaaggtgttgaaggcctggatatcggcaccaacgtggtggtttatggtccgtggggttgcggtaattgctggcattgtagccagggcctggaaaactattgtagccgtgcgcaggaactgggcatcaatccgccgggtctgggtgcgccgggtgcgctggcggaattcatgattgtggatagcccgcgtcatctggttccgattggcgatctggatccggtgaaaaccgttccgctgaccgatgcgggtctgaccccgtatcatgcgatcaaacgcagcctgccgaaactgcgtggtggtagctatgcggtggttattggcaccggtggtctgggtcatgttgcgattcagctgctgcgtcatctgagcgcggcgaccgttattgcgctggatgtgagcgcggataaactggaactggcgaccaaagttggtgcgcatgaagtggtgctgagcgataaagatgcggcggaaaacgtgcgcaaaatcaccggtagccagggtgcggcgctggttctggattttgtgggctaccagccgaccattgataccgcgatggcggttgcgggtgttggtagcgatgtggcgattgtgggcattggtgatggtcaggcgcatgcgaaagtgggtttcttccagagcccgtatgaagcgagcgttaccgttccgtattggggtgcgcgcaacgaactgattgaactgatcgatctggcgcatgcgggcatctttgatatcgcggtggaaaccttcagcctggataatggcgcggaagcgtatcgtcgtctggcggcgggcaccctgagcggtcgtgcggttgttgttccgggtctgtaagcttctgcatcgatcaccacaattcagcaaattgtgaacatcatcacgttcatctttccctggttgccaatggcccattttcctgtcagtaacgagaaggtcgcgaattcaggcgctttttagactggtcgtaatgaacaattcttaagaaggagatataca

(19) United States
(12) Patent Application Publication
(10) Pub. No.: US 2008/0145904 A1
(43) Pub. Date: Jun. 19, 2008

(54) Method For Producing Primary Alcohols

(76) Inventors: Harald Groger, Erlangen (DE); Francoise Chamouleau, Hanau (DE); Chad Hagedorn, Bellbrook, OH (US)

Correspondence Address: FULBRIGHT & JAWORSKI, LLP, 666 FIFTH AVE, NEW YORK, NY, 10103-3198, US

(21) Appl. No.: 11/659,291

(22) PCT Filed: Aug. 4, 2005
(86) PCT No.: PCT/EP05/08471

§ 371 Date: Apr. 2, 2007

(30) Foreign Application Priority Data
Aug. 5, 2004 (DE) 10 2004 038 054.6

(51) Int. Cl. C12P 7/04

(52) U.S. Cl. 435/157

(57) ABSTRACT

The present invention relates to a process for the production of primary alcohols from aldehydes with the said of whole-cell catalysts or isolated enzymes. Employed are an alcohol dehydrogenase and an enzyme which is capable of regenerating the cofactor, where it is preferred to have a substrate concentration of >150 mM aldehyde for the conversion.

The present invention relates to a process for the production of primary alcohols starting from aldehydes. The reduction is carried out by means of cofactor-dependent oxidoreductases, the cofactor, in turn, being regenerated again by a second enzymatic system.

The production of primary alcohols is of interest for the food industry because these products are used as aroma chemicals, either in direct form or after having been converted into corresponding ester compounds. A preferred way of producing them is to obtain the primary alcohols by reducing aldehydes. This reduction could, in principle, be effected by using nonnatural chemical reducing agents, as described in a large number of existing protocols in the literature, for example by using metal hydrides. However, such a route would only give “nature-identical”, but not “natural” primary alcohols. However, it is precisely the food industry where obtaining natural primary alcohols is of great importance. One route of producing them consists in enzymatic reduction of aldehydes.

In principle, the enzymatic reduction of aldehydes has already been described extensively in the literature. In particular, examples are known for the production of trans-3-hexenol, trans-2-hexenol, cinnamic alcohol and 2-phenylethanol as the result of these substances being used as aroma chemicals or as intermediates for aroma chemicals in the food industry sector. However, the reactions are carried out in highly dilute media, which, technically speaking, has the disadvantage of the low product concentrations which this entails.

The reduction of cinnamaldehyde with a substrate concentration of 0.05 g per liter of reaction volume in the presence of Botrytis cinerea strains is described by G. Bock et al. (G. Bock et al., Z. Lebensm. Unters. Forsch. 1988, 186, 33-35). The highest yields were 0.019 and 0.025 g per liter of reaction volume.

The utilization of alcohol dehydrogenases for the production of 2-phenylethanol by reduction reactions is also described. Typically, the product concentrations obtained under standard batch conditions are just <1 g/l (M. W. T. Etschmann, D. Sell, J. Schrader, Biotechnol. Lett. 2003, 25, 531-536). Employing an in-situ product removal technique has resulted in an increase to 2 g/l, oleyl alcohol being required as auxiliary compound in a further, second phase (M. W. T. Etschmann, D. Sell, J. Schrader, Biotechnol. Lett. 2003, 25, 531-536). Using an extractive fed-batch biotransformation with oleic acid as further, organic phase, Stark et al. successfully used such a specific reaction system for obtaining 2-phenylethanol at a product concentration of 12.6 g/l, which corresponds to an amount of approx. 100 mM (D. Stark, T. Munch, B. Sonnleitner, I. W. Marison, U. von Stockar, Biotechnol. Prog. 2002, 18, 514-523).

A series of enzymatic processes which, as final key step, comprise in each case an enzymatic reduction of the corresponding aldehyde have been described for the production of trans-3-hexenol and trans-2-hexenol (S. K. Goers et al., U.S. Pat. No. 4,806,379, 1989; B. Muller et al., U.S. Pat. No. 5,464,761, 1995; P. Brunerie et al., U.S. Pat. No. 5,620,879, 1997; M.-L. Fauconnier et al., Biotechnology Lett. 1999, 21, 629-633; R. B. Holtz et al., U.S. Pat. No. 6,274,358, 2001). The highest yields were reported by Muller et al. with 4.2 g per kg in the case of trans-3-hexenol and 1.5 g per kg in the case of trans-2-hexenol (B. Muller et al., U.S. Pat. No. 5,464,761, 1995; see also: J. Schrader et al., Biotechnology Letters 2004, 26, 463-472). The disadvantages in these processes are mainly the low substrate concentrations used in the procedure and the resulting low product concentrations obtained, which are <5 g per kg of reaction solution.

To avoid this disadvantage, suitable enzyme reactions with trans-2-hexenal were carried out at substrate concentrations of 100 mM (Example 4=comparative example). The enzyme employed here was an alcohol dehydrogenase from Rhodococcus erythropolis, after these enzymes had been found to have an activity for trans-2-hexenal. The cofactor regeneration was effected with a formate dehydrogenase, after this cofactor-generating enzyme had been employed successfully in a number of cases in the enzymatic reduction of ketones (M.-R. Kula, U. Kragl, Dehydrogenases in the Synthesis of Chiral Compounds in: Stereoselective Biocatalysis (Ed.: R. N. Patel), Marcel Dekker, New York, 2000, chapter 28, p. 839 et seq.). In the presence of the two enzyme components, however, the desired reaction was only observed to a small degree, with a conversion of only 16% after 72 hours (Example 4), probably as the result of inhibitory and/or destabilizing effects owing to already low trans-2-hexenal concentrations.

M.-L. Fauconnier et al. also report on the fact that trans-3-hexenal inhibits alcohol dehydrogenases when employing alcohol-dehydrogenase-containing micro-organisms, even at very low substrate concentrations (M.-L. Fauconnier et al., Biotechnology Lett. 1999, 21, 629-633). Typically, the reactions were carried out at 0.67 mM (!), corresponding to 0.066 g/l. The maximum conversion rate was 82%.

In short, all enzymatic reductive processes of aldehydes for the purpose of producing primary alcohols currently only lead to low, technically unattractive product concentrations of <15 g/l. Correspondingly, only low substrate concentrations of not more than 100 mM give technically meaningful conversion rates of >80%. The root cause therefor can be explained as the aldehyde component having an inhibitory effect and the enzymes being deactivated. Since aldehydes are much more reactive components in comparison with ketones, these compounds can substantially react with functional groups (in particular amines) of the enzymes, which is undesired, thus deactivating them.

It was therefore an object of the present invention to develop a rapid, simple, inexpensive and effective enzymatic process for the production of primary alcohols from aldehydes. In particular, it was an aim to improve the space-time yield over the known prior-art processes. This necessitates firstly carrying out the process at high substrate concentrations with correspondingly good yields in a robust procedure. In particular, it should be possible to employ such a process for reducing unsaturated aldehydes.

The object was solved according to the claims. Claim 1 relates to a process according to the invention in which a rec whole-cell catalyst is employed. Claim 2 comprises a preferred embodiment of this process. Claim 3 is directed at the use of the free enzymes for the purpose according to the invention. Claims 3 to 9 protect preferred embodiments of the process according to the invention.

Owing to the fact that, in a process for the production of primary alcohols by reducing aldehydes, this conversion is carried out in the presence of a recombinant whole-cell catalyst comprising an alcohol dehydrogenase and an enzyme capable of regenerating the cofactor, one arrives entirely surprisingly, but no less advantageously, at the solution of the problem posed. Surprisingly, high to very high conversion rates are obtained using this process at high substrate concentrations of >150 mM, in particular >250 mM and very preferably >500 mM. These conversion rates are typically above 80% and in particular greater than 90-95% yield. Based on the low conversion rates of previous processes, this could not have been expected, even when using low substrate concentrations, and is extremely surprising in particular against the background of the severe inhibition or destabilization effects which the substrates employed exert on dehydrogenases, a fact known from the prior art.

In principle, all alcohol dehydrogenases which a person skilled in the art might conceive for the present application are suitable for establishing the rec whole-cell catalyst. However, the conversion according to the invention was preferably carried out in the presence of a recombinant (rec) whole-cell catalyst comprising an alcohol dehydrogenase and an enzyme capable of regenerating the cofactor from the group of the glucose dehydrogenases or malate dehydrogenases.

A further solution of the problem posed above is achieved by carrying out the conversion according to the invention in the presence of an isolated alcohol dehydrogenase and an isolated enzyme capable of regenerating the cofactor from the group of the glucose dehydrogenases or malate dehydrogenases. What is surprising here is the specific suitability of the combination of an alcohol dehydrogenase with an enzyme capable of regenerating the cofactor from the group of the glucose dehydrogenases or malate dehydrogenases in the form of enzymes employed in isolated form, in particular taking into consideration that the yields are not satisfactory when formate dehydrogenase is employed in this manner (see also Example 4=comparative example).

The markedly improved performance of the combination of an alcohol dehydrogenase with an enzyme capable of regenerating the cofactor from the group of the glucose dehydrogenases or malate dehydrogenases over the analogous combination with a formate dehydrogenase is illustrated for example by comparing the conversion rates obtained in the reduction of trans-2-hexenal (see Example 4 (=comparative example) and Examples 5 to 7).

The conversion is preferably carried out at high substrate concentrations of >150 mM, in particular >250 mM and very preferably >500 mM of aldehyde.

In accordance with the invention, the term “at high substrate concentrations of >150 mM” is understood as meaning that >150 mM of the substrate are converted per starting volume of aqueous solvent employed (including buffer system) when using the process described. In this context, it is not critical whether the >150 mM of substrate as concentration in the reaction mixture are indeed achieved or whether a substrate concentration of >150 mM is converted in total, based on the starting volume of aqueous solvent.

A very especially preferred variant is, however, one in which a substrate concentration of >150 mM and the like of aldehyde is indeed provided for the conversion. The concentrations detailed herein refer to substrate (aldehyde) concentrations which are indeed obtained in the reaction mixture, based on the starting volume of aqueous solvent, where it is irrelevant when during the incubation period of a whole-cell catalyst employed or of isolated enzymes employed (in purified or partially purified form or as crude extract) this starting concentration is achieved. It is only achieved at least once.

When using a whole-cell catalyst, it is possible to employ the aldehyde directly at the beginning of a whole-cell reaction at these concentrations in the form of a batch, or else it is possible first to use a whole-cell catalyst up to a certain optical density before adding the aldehyde. Likewise, the aldehyde can first be employed at lower concentrations and then added during the incubation period of the cell reaction up to concentrations as stated. It is preferred, however, if a substrate concentration of >150 mM and the like can be achieved in the reaction at least once during the conversion of the substrate to the desired alcohol.

All aldehydes can be employed as the aldehyde component. Preferably, the aldehyde component employed can be subsumed under the following general structural formula

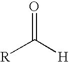

in which R is (C₁-C₂₀)-alkyl, (C₂-C₂₀)-alkenyl, (C₂-C₂₀)-alkynyl, (C₁-C₂₀)-alkoxy, HO— (C₁-C₂₀)-alkyl, (C₂-C₂₀)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₂₀)-alkyl-(C₆-C₁₈)-aryl, (C₁-C₂₀)-alkyl-(C₃-C₁₈)-heteroaryl, (C₃-C₈)-cycloalkyl, (C₁-C₂₀)-alkyl-(C₃-C₈)-cycloalkyl, (C₃-C₈)-cycloalkyl-(C₁-C₂₀)-alkyl, (C₆-C₁₈)-aryl-(C₂-C₂₀)-alkenyl. The process is used in particular for reducing aldehydes which comprise at least one C═C double bond in the moiety. Substances which are very preferably employed here are 2-trans-hexenal, 2-cis-hexenal, 3-trans-hexenal, 3-cis-hexenal and/or cinnamaldehyde.

For the present invention, one of the enzymes preferably to be selected is an alcohol dehydrogenase. The skilled worker is not bound in his choice of alcohol dehydrogenase. Alcohol dehydrogenases which have proved to be preferred are, for example, alcohol dehydrogenases from a Lactobacillus strain, in particular from Lactobacillus kefir and Lactobacillus brevis, or alcohol dehydrogenases from a Rhodococcus strain, in particular from Rhodococcus erythropolis and Rhodococcus ruber, or alcohol dehydrogenases from an Arthrobacter strain, in particular from Arthrobacter paraffineus.

Preferred dehydrogenases for cofactor regeneration have proved to be glucose dehydrogenases, preferably a glucose dehydrogenase from Bacillus, Thermoplasma and Pseudomonas strains. Glucose dehydrogenases are described for example by A. Bommarius in: Enzyme Catalysis in Organic Synthesis (Ed.: K. Drauz, H. Waldmann), Volume III, Wiley-VCH, Weinheim, 2002, chapter 15.3.

Malate dehydrogenases are known to the skilled worker (S.-I. Suye, M. Kawagoe, S. Inuta, Can. J. Chem. Eng. 1992, 70, 306-312; S.-I. Suye, Recent Res. Devel. Ferment. Bioeng. 1998, 1, 55-64; PhD thesis S. Naamnieh, University of Düsseldorf; WO2004/022764). Again, the skilled worker will select the dehydrogenase which can be employed most efficiently for his purpose. In principle, preferred malate dehydrogenases are those which regenerate NAD(P)H to such an extent that no bottleneck occurs for the course of the reaction of the other enzyme employed. Malate dehydrogenases (“malic enzyme”) employed are preferably a “malic enzyme” from Sulfolobus, Clostridium, Bacillus and Pseudomonas strains and from E. coli. Very preferred in this context is the E. coli K12 malate dehydrogenase, which is known. Gene isolation and cloning are described in S. Naamnieh, PhD thesis, University of Düsseldorf, p. 70 et seq.

The aldehyde can be added in any manner. Preferably, all of the aldehyde is added at the beginning (batch reaction) or, as alternative, metered in. A continuous adding (continuous feed-in method) may also be employed. These procedures are well known to the skilled worker and are employed analogously in the present case.

In accordance with the present invention, a “recombinant whole-cell catalyst” is understood as meaning a cell in which at least one recombinant gene is expressed or has been expressed, that is to say at east one recombinant protein is present which is capable of catalyzing the conversion according to the invention (reduction of the aldehyde and/or regeneration of the cofactor). The recombinant proteins are not limited to a presence in live or nonlive whole-cell catalysts, but can be in any active form. In this context, “active form” is understood as meaning the ability of the recombinant protein of catalyzing an enzymatic reaction. In a preferred embodiment of the present invention, the recombinant protein is distributed over the cytosol of the cell in exclusively free form and is not present in the form of inclusion bodies. In accordance with the invention, the cell is, or has been, capable of expressing an alcohol dehydrogenase and a dehydrogenase capable of regenerating the cofactor.

All known cells are suitable as whole-cell catalyst comprising an alcohol dehydrogenase and an enzyme capable of regenerating the cofactor. Microorganisms to be mentioned in this context are organisms such as, for example, yeasts such as Hansenula polymorpha, Pichia sp., Saccharomyces cerevisiae, prokaryotes such as E. coli, Bacillus subtilis or eukaryotes such as mammalian cells, insect cells or plant cells. The cloning methods are well known to a person skilled in the art (Sambrook, J.; Fritsch, E. F. and Maniatis, T. (1989), Molecular cloning: a laboratory manual, 2^{nd} ed., Cold Spring Harbor Laboratory Press, New York). E. coli strains are preferably to be employed for this purpose. Very especially preferred are: E. coli XL1 Blue, NM 522, JM101, JM109, JM105, RR1, DH5α, TOP 10-, HB101, BL21 codon plus, BL21 (DE3) codon plus, BL21, BL21 (DE3), MM294. Plasmids with which the gene construct containing the nucleic acid according to the invention is preferably cloned into the host organism are also known to the skilled worker (see also PCT/EP03/07148; see hereinbelow).

Plasmids or vectors which are suitable are, in principle, all of the embodiments available to the skilled worker for this purpose. Such plasmids and vectors can be found for example in Studier and coworkers (Studier, W. F.; Rosenberg A. H.; Dunn J. J.; Dubendroff J. W.; (1990), Use of the T7 RNA polymerase to direct expression of cloned genes, Methods Enzymol. 185, 61-89) or the catalogs of Novagen, Promega, New England Biolabs, Clontech or Gibco-BRL. Further preferred plasmids and vectors can be found in: Glover, D. M. (1985), DNA cloning: a practical approach, Vol. I-III, IRL Press Ltd., Oxford; Rodriguez, R. L. and Denhardt, D. T. (eds) (1988), Vectors: a survey of molecular cloning vectors and their uses, 179-204, Butterworth, Stoneham; Goeddel, D. V. (1990), Systems for heterologous gene expression, Methods Enzymol. 185, 3-7; Sambrook, J.; Fritsch, E. F. and Maniatis, T. (1989), Molecular cloning: a laboratory manual, 2^{nd} ed., Cold Spring Harbor Laboratory Press, New York.

Plasmids by means of which the gene constructs containing the nucleic acid sequences considered can be cloned into the host organism in a very especially preferred manner are, or are based on: pUC18/19 (Roche Biochemicals), pKK-177-3H (Roche Biochemicals), pBTac2 (Roche Biochemicals), pKK223-3 (Amersham Pharmacia Biotech), pKK-233-3 (Stratagene) or pET (Novagen).

In a further embodiment of the process according to the invention, the whole-cell catalyst is preferably pretreated before being used in such a manner that the permeability of the cell membrane for the substrates and products is increased over the intact system. Especially preferred in this context is a process in which the whole-cell catalyst is pretreated for example by freezing and/or treatment with an organic solvent, in particular toluene.

Particularly suitable in this context is a recombinant whole-cell catalyst which contains an alcohol dehydrogenase from R. erythropolis or Lactobacillus kefir and a malate dehydrogenase (including what are known as malic enzymes). Equally suitable are recombinant whole-cell catalysts with an alcohol dehydrogenase from R. erythropolis or Lactobacillus kefir and a glucose dehydrogenase from Thermoplasma acidophilum or Bacillus subtilis in all possible combinations. The two recombinant whole-cell catalysts which are described in the experimental part are preferred as especially suitable recombinant whole-cell catalysts.

The concentration of this recombinant whole-cell catalyst is preferably not more than 75 g/l, in a further preferred embodiment up to 50 g/l, very especially preferably up to 25 g/l and particularly preferably up to 15 g/l, g referring to wet biomass (WBM).

The process according to the invention can be carried out at any reaction temperatures, in particular those which are suitable for the recombinant whole-cell catalyst used. A reaction temperature to be considered as being particularly suitable is a reaction temperature of from 10 to 90° C., preferably 15 to 50° C. and especially preferably 20 to 35° C.

As regards the pH of the reaction, the skilled worker is again free to choose, it being possible for the reaction to be carried out at a fixed pH or else while varying the pH within a pH interval. The pH is selected in particular taking into consideration the requirements of the host organism or the isolated enzymes employed. Preferably, the reaction is carried out at a pH at 5 to 9, preferably pH 6 to 8 and especially preferably at pH 6.5 to 7.5.

The conversion of the substrate employed to give the desired product is preferably carried out in a cell culture, using a suitable recombinant whole-cell catalyst. Depending on the host organism used, a suitable nutrient medium is used for this purpose. The media which are suitable for the host cells are generally known and commercially available. Moreover, customary additions can be added to the cell cultures, such as, for example, antibiotics, growth-promoting agents such as, for example, sera (fetal calf serum and the like) and similar, known additives.

In a preferred embodiment, the conversion of the aldehyde to give the desired primary alcohol is carried out without addition of an organic solvent. This means that no organic solvent is added to the reaction mixture which contains the biocatalyst. Alternatively, however, it is possible to add further organic solvents to the added water required for carrying out the reaction, preferably organic solvents which are soluble in water. These are taken to mean, in particular, water-soluble organic solvents such as, for example, alcohols, in particular methanol or ethanol, or ethers such as THF or dioxane.

Moreover, it is preferred to carry out the conversion in a cell suspension of the suitable recombinant whole-cell catalyst, it being possible for the aldehyde employed in the cell suspension also to be present as a suspension, or to be present in the form of an emulsion or solution in the cell suspension.

When using isolated enzymes (in purified or partially purified form or as crude extracts or in immobilized form), a preferred embodiment entails adding the corresponding cofactor in suitable amounts. Typically, the added amounts of cofactor are in the range of from 0.00001 to 0.1 equivalents, preferably 0.0001 to 0.01 and very especially 0.0001 and 0.001 equivalents. In a preferred embodiment, and when employing a whole-cell catalyst, the use of an “external” cofactor addition can be dispensed with, or such an “external” cofactor addition can be used in a range of less than 0.0005 equivalents.

For the present reaction, a procedure is followed in a preferred embodiment in which the recombinant whole-cell catalyst, or the isolated enzymes, and the substrate are initially introduced into the chosen solvent system. Then, a certain amount of the corresponding cofactor required (for example NADH or NADPH, or their oxidized forms NAD⁺ and NADP⁺) can be added to the reaction mixture, as required. However, the order of the addition can be varied. The reaction mixture is worked up by methods known to the skilled worker. In the case of a batch process, the biomass can be separated readily from the product by means of filtration or centrifugation. The alcohol obtained can then be isolated by customary methods (for example extraction, distillation, crystallization).

However, the present process can also be carried out continuously. To this end, the reaction is carried out in what is known as an enzyme membrane reactor in which high-molecular-weight substances—the enzymes or biomass—are retained behind an ultrafiltration membrane and low-molecular-weight substances—such as the amino acids produced—can pass across the membrane. Such a procedure has already been described repeatedly in the prior art (Wandrey et al. in Jahrbuch 1998, Verfahrenstechnik und Chemieingenieurwesen, VDI, p. 151 et seq.; Kragl et al., Angew. Chem. 1996, 6, 684).

(C₁-C₂₀)-Alkyl radicals are in particular methyl, ethyl, n-propyl, isopropyl, n-butyl, isobutyl, sec-butyl, tert-butyl, pentyl, hexyl, heptyl, octyl, nonyl, decyl, undecyl, dodecyl and all their bonding isomers. A (C₁-C₈)-alkyl radical is, analogously, one in which 1 to 8 C atoms are present in the chain.

(C₂-C₂₀)-Alkenyl radical means a (C₁-C₂₀)-alkyl radical as described above with at least one C═C double bond. (C₂-C₂₀)-Alkynyl radical means a (C₁-C₂₀)-alkyl radical as described above with at least one C≡C triple bond. The (C₁-C₂₀)-alkoxy radical corresponds to the (C₁-C₂₀)-alkyl radical, with the proviso that the latter is bonded to the molecule via an oxygen atom. The same applies analogously to a (C₁-C₈)-alkoxy radical.

(C₂-C₂₀)-Alkoxyalkyl is understood as meaning radicals in which the alkyl chain is interrupted by at least one oxygen function, it not being possible for two oxygen atoms to be linked with one another. The number of carbon atoms indicates the total number of carbon atoms present in the radical.

The above-described radicals can be monosubstituted or polysubstituted by halogens and/or by radicals containing N, O, P, S, Si atoms. They are in particular alkyl radicals of the abovementioned type which contain one or more of these heteroatoms within their chain or which are bonded to the molecule via one of these heteroatoms.

(C₃-C₈)—Cycloalkyl is understood as meaning cyclopropyl, cyclobutyl, cyclopentyl, cyclohexyl or cycloheptyl residues and the like. They can be substituted by one or more halogens and/or radicals containing N, O, P, S, Si atoms and/or can contain N, O, P, S atoms within the ring, such as, for example, 1-, 2-, 3-, 4-piperidyl, 1-, 2-, 3-pyrrolidinyl, 2-, 3-tetrahydrofuryl, 2-, 3-, 4-morpholinyl.

A (C₃-C₈)-cycloalkyl-(C₁-C₂₀)-alkyl radical denotes a cycloalkyl radical as described above which is bonded to the molecule via an alkyl radical as stated above.

For the purposes of the invention, (C₁-C₈)-acyloxy means an alkyl radical as defined above with not more than 8 carbon atoms which is bonded to the molecule via a CO function.

For the purposes of the invention, (C₁-C₈)-acyl means an alkyl radical as defined above with not more than 8 carbon atoms which is bonded to the molecule via a CO function.

A (C₆-C₁₈)-aryl radical is understood as meaning an aromatic radical with 6 to 18 C atoms. This includes, in particular, compounds such as phenyl, naphthyl, anthryl, phenanthryl, biphenyl radicals or systems of the previously described type which are condensed with the molecule in question, such as, for example, indenyl systems which can optionally be substituted by (C₁-C₈)-alkyl, (C₁-C₈)-alkoxy, NR¹R², (C₁-C₈)-acyl, (C₁-C₈)-acyloxy.

A (C₇-C₂₆)-aralkyl radical is a (C₆-C₁₈)-aryl radical which is bonded to the molecule via a (C₁-C₈)-alkyl radical.

For the purposes of the invention, a (C₃-C₁₈)-heteroaryl radical denotes a five-, six- or seven-membered aromatic ring system of 3 to 18 C atoms which has heteroatoms such as, for example, nitrogen, oxygen or sulfur in the ring. Such heteroaromatics are, in particular, considered to be radicals such as 1-, 2-, 3-furyl, such as 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl.

A (C₄-C₂₆)-heteroaralkyl is understood as meaning a heteroaromatic system which corresponds to the (C₇-C₂₆)-aralkyl radical.

Halogens (Hal) which are suitable are fluorine, chlorine, bromine and iodine.

The term “isolated enzyme” is understood as meaning, in accordance with the invention, the use of the alcohol dehydrogenase and of the enzyme capable of regenerating the cofactor from the group of the glucose dehydrogenases or malate dehydrogenases as isolated enzymes (in purified or partially purified form or as a crude extract or in immobilized form).

The malate dehydrogenase (MDH), referred to as “malic enzyme”, catalyzes the oxidative decarboxylation of malate to pyruvate. A large number of malate dehydrogenases from various organisms are known, thus, inter alia, from higher animals, plants and microorganisms. One distinguishes between four types of malate dehydrogenases, which are classified in the enzyme classes E.C. 1.1.1.37 to EC 1.1.1.40 (http://www.genome.ad.jp). Depending on the type of malate dehydrogenase, NAD and/or NADP is/are required as cofactor.

The E. coli used in the examples has been deposited by the applicant at the DSMZ GmbH, Mascheroder Weg 1b, D-38124 Brunswick on 08.24.01 under the number DSM 14459 in accordance with the Budapest Treaty.

### FIGURES

FIG. 1 shows the plasmid map of plasmid pNO5c

FIG. 2 shows the plasmid map of plasmid pNO8c

FIG. 3 shows the plasmid map of plasmid pNO14c

### EXPERIMENTAL PART

### Preparation of a Whole-Cell Catalyst Comprising an (R)-Alcohol Dehydrogenase from Lactobacillus kefir and a Glucose Dehydrogenase from Thermoplasma acidophilum

### Strain Production

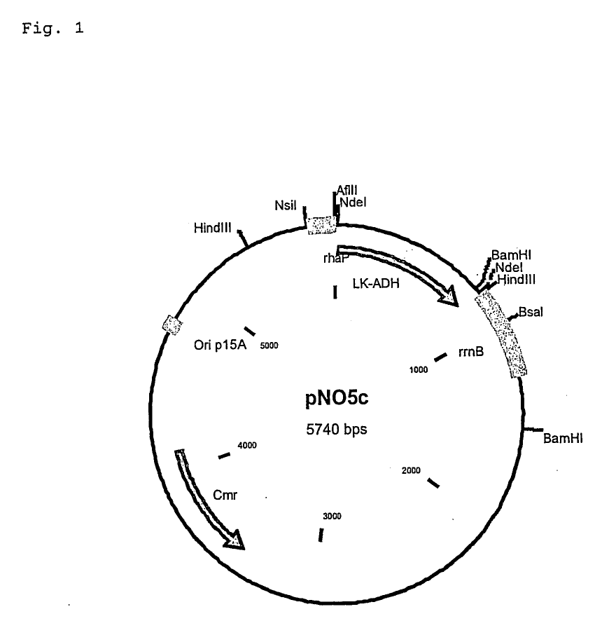

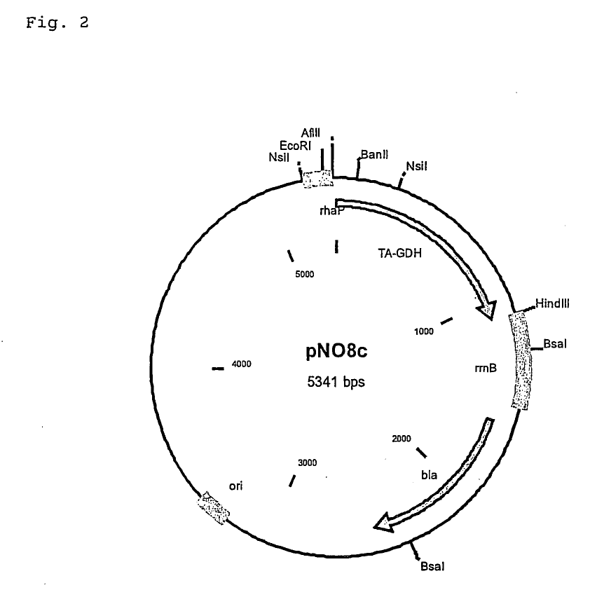

Chemically competent cells of E. coli DSM14459 (described in the patent WO03/042412) were transformed with the plasmid pNO5c (Sambrook et al. 1989, Molecular cloning: A Laboratory Manual, 2nd Edition, Cold Spring Harbor Laboratory Press). This plasmid codes for the alcohol dehydrogenase from Lactobacillus kefir (Lactobacillus kefir alcohol dehydrogenase: a useful catalyst for synthesis. Bradshaw et al. JOC 1992, 57 1532-6, Reduction of acetophenone to R(+)-phenylethanol by a new alcohol dehydrogenase from Lactobacillus kefir. Hummel W. Ap Microbiol Biotech 1990, 34, 15-19). The recombinant E. coli DSM14459 (pNO5c—FIG. 1) was made chemically competent and transformed with the plasmid pNO8c (FIG. 2), which codes for the gene of a codon-optimized glucose dehydrogenase from Thermoplasma acidophilum (Bright, J. R. et al., 1993 Eur. J. Biochem. 211:549-554). Both genes are under the control of a rhamnose promoter (Stumpp, Tina; Wilms, Burkhard; Altenbuchner, Josef. A new, L-rhamnose-inducible expression system for Escherichia coli. BIOspektrum (2000), 6(1), 33-36). Sequences and plasmid maps of pNO5c and pNO8c are detailed hereinbelow.

### Preparation of Active Cells

A single colony of E. coli DSM14459 (pNO5c, pNO8c) was incubated for 18 hours at 37° C. in 2 ml of LB medium with added antibiotics (50 μg/l ampicillin and 20 μg/ml chloramphenical) while shaking (250 rpm). This culture was diluted 1:100 in fresh LB medium with rhamnose (2 g/l) as inductor, added antibiotics (50 μg/l ampicillin and 20 μg/ml chloramphenical) and 1 mM ZnCl₂ and incubated for 18 hours at 30° C., with shaking (250 rpm). Cells were centrifuged (10 000 g, 10 min, 4° C.), the supernatant was discarded, and the cell pellet was employed in biotransformation experiments, either directly or after storage at −20° C.

### Preparation of a Whole-Cell Catalyst Comprising an (S)-Alcohol Dehydrogenase from Rhodococcus erythropolis and a Glucose Dehydrogenase from Bacillus subtilis

### Strain Production

Chemically competent cells of E. coli DSM14459 (described in the patent WO03/042412) were transformed with the plasmid pNO14c (Sambrook et al. 1989, Molecular cloning: A Laboratory Manual, 2nd Edition, Cold Spring Harbor Laboratory Press). This plasmid codes for an alcohol dehydrogenase from Rhodococcus erythropolis (Cloning, sequence analysis and heterologous expression of the gene encoding a (S)-specific alcohol dehydrogenase from Rhodococcus erythropolis DSM 43297. Abokitse, K.; Hummel, W. Applied Microbiology and Biotechnology 2003, 62 380-386) and a glucose dehydrogenase from Bacillus subtilis (Glucose dehydrogenase from Bacillus subtilis expressed in Escherichia coli. I: Purification, characterization and comparison with glucose dehydrogenase from Bacillus megaterium. Hilt W; Pfleiderer G; Fortnagel P Biochimica et biophysica acta (1991 Jan. 29), 1076(2), 298-304). The alcohol dehydrogenase is under the control of a rhamnose promoter (Stumpp, Tina; Wilms, Burkhard; Altenbuchner, Josef. A new, L-rhamnose-inducible expression system for Escherichia coli. BIOspektrum (2000), 6(1), 33-36). The sequence and plasmid map of pNO14c is detailed hereinbelow.

### Preparation of Active Cells

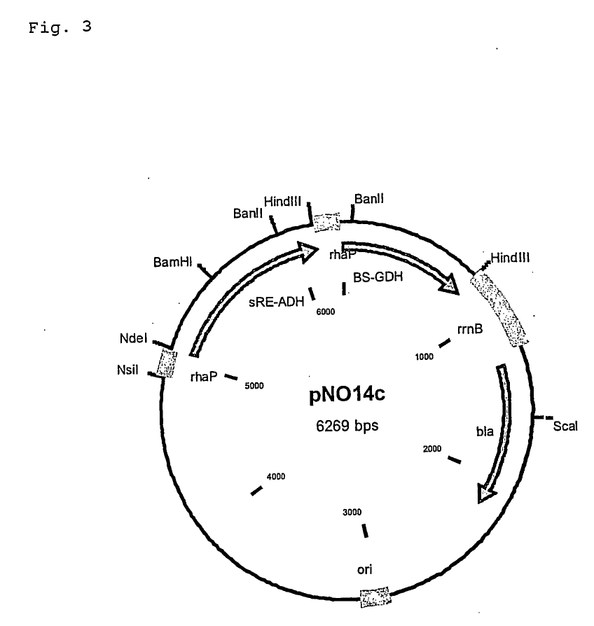

A single colony of E. coli DSM14459 (pNO14c—FIG. 3) was incubated for 18 hours at 37° C. in 2 ml of LB medium with added antibiotics (50 μg/l ampicillin and 20 μg/ml chloramphenical) while shaking (250 rpm). This culture was diluted 1:100 in fresh LB medium with rhamnose (2 g/l) as inductor, added antibiotics (50 μg/l ampicillin and 20 μg/ml chloramphenical) and 1 mM ZnCl₂ and incubated for 18 hours at 30° C., with shaking (250 rpm). Cells were centrifuged (10 000 g, 10 min, 4° C.), the supernatant was discarded, and the cell pellet was employed in biotransformation experiments, either directly or after storage at −20° C.

Examples of the production of cinnamic alcohol and trans-2-hexen-1-ol:

### Example 1

### Reduction of Cinnamaldehyde in a 0.2 M Solution Using a Whole-Cell Catalyst Comprising an Alcohol Dehydrogenase and Glucose Dehydrogenase

In a Titrino reaction vessel, 40 ml of a phosphate buffer (brought to Ph 7.0) are treated, at room temperature, with the above-described whole-cell catalyst E. coli DSM14459 (pNO14c) with an alcohol dehydrogenase (E. coli, (S)-alcohol dehydrogenase from R. erythropolis, glucose dehydrogenase from B. subtilis) at such a cell concentration that an optical density of OD=24 is obtained, 1.05 equivalents of glucose (equivalents are based on amount of cinnamaldehyde employed) and 8 mmol of cinnamaldehyde (corresponding to a substrate concentration, based on phosphate buffer employed, of 0.2 M). The reaction mixture is stirred at room temperature, the pH being kept constant (at pH 6.5) by addition of sodium hydroxide solution (2 M NaOH). Samples are taken at regular intervals, and the conversion of the cinnamaldehyde into cinnamyl alcohol is determined by means of HPLC. After 1 hour, the conversion rates had reached 93%, and after 2 hours 100%.

### Example 2

### Reduction of Cinnamaldehyde in a 0.5 M Solution Using a Whole-Cell Catalyst Comprising an Alcohol Dehydrogenase and Glucose Dehydrogenase

In a Titrino reaction vessel, 40 ml of a phosphate buffer (brought to pH 7.0) are treated, at room temperature, with the above-described whole-cell catalyst E. coli DSM14459 (pNO14c) with an alcohol dehydrogenase (E. coli, (S)-alcohol dehydrogenase from R. erythropolis, glucose dehydrogenase from B. subtilis) at such a cell concentration that an optical density of OD=16 is obtained, 1.05 equivalents of glucose (equivalents are based on amount of cinnamaldehyde employed) and 20 mmol of cinnamaldehyde (corresponding to a substrate concentration, based on phosphate buffer employed, of 0.5 M). The reaction mixture is stirred at room temperature for 25 h, the pH being kept constant (at pH 6.5) by addition of sodium hydroxide solution (2 M NaOH). Samples are taken at regular intervals, and the conversion of the cinnamaldehyde into cinnamyl alcohol is determined by means of HPLC. After 1 hour, the conversion rates had reached 44%, after 5 hours 91% and after 25 hours 93%.

### Example 3

### Reduction of Cinnamaldehyde in a 1.5 M Solution Using a Whole-Cell Catalyst Comprising an (R)-Selective Alcohol Dehydrogenase

In a Titrino reaction vessel, 40 ml of a phosphate buffer (brought to pH 7.0) are treated, at room temperature, with the above-described whole-cell catalyst E. coli DSM14459 (pNO5c, pNO8c) with an (R)-selective alcohol dehydrogenase (E. coli, (R)-alcohol dehydrogenase from L. kefir, glucose dehydrogenase from T. acidophilum) at such a cell concentration that an optical density of OD=30 is obtained, 1.05 equivalents of glucose (equivalents are based on amount of cinnamaldehyde employed) and 60 mmol of cinnamaldehyde (corresponding to a substrate concentration, based on phosphate buffer employed, of 1.5 M). The reaction mixture is stirred at room temperature, the pH being kept constant by addition of sodium hydroxide solution (5 M NaOH). Samples are taken at regular intervals, and the conversion of the cinnamaldehyde into cinnamyl alcohol is determined by means of HPLC. After 1 hour, the conversion rates had reached 15%, after 5 hours 58% and after 23.5 hours 1098%.

### Example 4

### COMPARATIVE EXAMPLE

### Conversion of Trans-2-Hexenal at 100 mM Using a Formate Dehydrogenase for Cofactor Regeneration

A reaction mixture consisting of trans-2-hexenal (100 mM) and NADH (3.5 mM, corresponding to 0.035 equivalents based on the aldehyde), sodium formate (455 mM, corresponding to 4.55 equivalents based on the aldehyde), with enzyme quantities of 20 U/mmol of an (S)-ADH from R. erythropolis (expr. in E. coli) and 20 U/mmol of a formate dehydrogenase from Candida boidinii, is stirred over a period of 72 hours in 1 ml of a phosphate buffer (100 mM; pH 7.0) at a reaction temperature of 30° C. Within this period, samples are taken, and the respective conversion rate is determined via HPLC. The conversion rates were 7% after 5 hours and 16% after 72 hours.

### Example 5

### Conversion of Trans-2-Hexenal at 100 Mm Using a Glucose Dehydrogenase for Cofactor Regeneration

A reaction mixture consisting of trans-2-hexenal (100 mM) and NADH (1.4 mM, corresponding to 0.014 equivalents based on the aldehyde), glucose (300 mM, corresponding to 3 equivalents based on the aldehyde), with enzyme quantities of 20 U/mmol of an (S)-ADH from R. erythropolis (expr. in E. coli) and 150 U/mmol of a glucose dehydrogenase from Bacillus sp., is stirred over a period of 72 hours in 1 ml of a phosphate buffer (100 mM; pH 7.0) at a reaction temperature of 30° C. Within this period, samples are taken, and the respective conversion rate is determined via HPLC. The conversion rates were 64% after 5 hours and 72% after 72 hours.

### Example 6

### Conversion of Trans-2-Hexenal at 100 Mm Using a Glucose Dehydrogenase for Cofactor Regeneration

A reaction mixture consisting of trans-2-hexenal (100 mM) and NADPH (1 mM, corresponding to 0.01 equivalents based on the aldehyde), magnesium chloride (5 mM), glucose (300 mM, corresponding to 3 equivalents based on the aldehyde), with enzyme quantities of 13 U/mmol of an (R)-ADH from L. kefir and 60 U/mmol of a glucose dehydrogenase from Thermoplasma acidophilum, is stirred over a period of 72 hours in 1 ml of a phosphate buffer (100 mM; pH 7.0) at a reaction temperature of 30° C. Within this period, samples are taken, and the respective conversion rate is determined via HPLC. The conversion rates were 40% after 1 hour, 79% after 5 hours and 86% after 72 hours.

### Example 7

### Reduction of Trans-2-Hexenal in a 0.5 M Solution Using a Whole-Cell Catalyst Comprising an Alcohol Dehydrogenase and Glucose Dehydrogenase

In a Titrino reaction vessel, 40 ml of a phosphate buffer (brought to pH 7.0) are treated, at room temperature, with the above-described whole-cell catalyst E. coli DSM14459 (pNO5c, pNO8c) containing an (R)-selective alcohol dehydrogenase (E. coli, (R)-alcohol dehydrogenase from L. kefir, glucose dehydrogenase from T. acidophilum) in such a cell concentration that an optical density of OD=27 is obtained, 6 equivalents of glucose (equivalents are based on amount of cinnamaldehyde employed) and 20 mmol of trans-2-hexenal (corresponding to a substrate concentration of 0.5 M based on phosphate buffer employed). The reaction mixture is stirred for 24 hours at room temperature, the pH being kept constant by addition of sodium hydroxide solution (2M NaOH). Samples are taken at regular intervals, and the conversion rate of the trans-2-hexenal into trans-2-hexen-1-ol is determined by means of HPLC. The conversion rates were 24% after 1 hour, 61% after 5 hours and >99% after 24 hours.

1-10. (canceled)

11. A process comprising producing a primary alcohol by reducing an aldehyde, in the presence of a recombinant whole-cell comprising an alcohol dehydrogenase and an enzyme capable of regenerating a cofactor for the alcohol dehydrogenase selected from the group consisting of a glucose dehydrogenase and a malate dehydrogenase at a substrate concentrations of >150 mM of aldehyde.

12. A process comprising producing a primary alcohol by reducing an aldehyde, in the presence of an isolated alcohol dehydrogenase and an isolated enzyme capable of regenerating a cofactor for the alcohol dehydrogenase selected from the group consisting of a glucose dehydrogenase and a malate dehydrogenase and in that the conversion is carried out at high substrate concentrations of >150 mM of aldehyde.

13. The process of claim 11, wherein the aldehyde is at least one of 2, characterized in that 2-trans-hexenal, 2-cis-hexenal, 3-trans-hexanal, 3-cis or cinnamaldehyde.

14. The process of claim 12, wherein the aldehyde is at least one of 2, characterized in that 2-trans-hexenal, 2-cis-hexenal, 3-trans-hexenal, 3-cis or cinnamaldehyde.

15. The process of claim 11, wherein the alcohol dehydrogenase is from Lactobacillus strain or from a Rhodococcus strain.

16. The process of claim 12, wherein the alcohol dehydrogenase is from Lactobacillus strain or from a Rhodococcus strain.

17. The process of claim 11, wherein the alcohol dehydrogenase is from Lactobacillus kefir, Lactobacillus brevis, or Rhodococcus erythropolis.

18. The process of claim 12, wherein the alcohol dehydrogenase is from Lactobacillus kefir, Lactobacillus brevis, or Rhodococcus erythropolis.

19. The process of claim 11, wherein the enzyme capable of regenerating the cofactor is a glucose dehydrogenase or a formate dehydrogenase.

20. The process of claim 11, wherein the enzyme capable of regenerating the cofactor is a glucose dehydrogenase from a Bacillus strain, a Pseudomonas strain, a Thermoplasma strain, Candida strain or a Pseudomonas strain.

21. The process of claim 12, wherein the enzyme capable of regenerating the cofactor is a glucose dehydrogenase from a Bacillus strain, a Pseudomonas strain, a Thermoplasma strain, Candida strain or a Pseudomonas strain.

22. The process as of claim 11, wherein the enzyme employed capable of regenerating the cofactor is a malate dehydrogenase.

23. The process of claim 12, wherein the enzyme employed capable of regenerating the cofactor is a malate dehydrogenase.

24. The process of claim 11, wherein E. coli is a host organism.

25. The process as claimed in claim 11, wherein >150 mM of the substrate is converted per starting volume employed of aqueous solvent.

26. The process as claimed in claim 12, wherein >150 mM of the substrate is converted per starting volume employed of aqueous solvent.

27. The process as claimed in claim 11, wherein a concentration of >150 mM of substrate is present in the reaction mixture.

28. The process as claimed in claim 12, wherein a concentration of >150 mM of substrate is present in the reaction mixture.

29. The process as claimed in claim 11, wherein a substrate concentration of in total >150 mM is converted, based on the starting volume of aqueous solvent.

30. The process as claimed in claim 12, wherein a substrate concentration of in total >150 mM is converted, based on the starting volume of aqueous solvent.
